(19) United States
(12) Patent Application Publication
(10) Pub. No.: US 2026/0370501 A1
(43) Pub. Date: Nov. 19, 2026

(54) MONITORING NETWORK

(71) Applicant: BRITISH TELECOMMUNICATIONS PUBLIC LIMITED COMPANY, London, Greater London (GB)

(72) Inventors: Albert RAFEL PORTI, London, Greater London (GB); Ian COOPER, London, Greater London (GB)

(21) Appl. No.: 19/153,830

(22) PCT Filed: Jan. 16, 2024
(86) PCT No.: PCT/EP2024/050902

§ 371 Date: Aug. 5, 2025

(30) Foreign Application Priority Data
Feb. 6, 2023 (EP) 23155086.4

(51) Int. Cl. H04L 12/12; G16H 40/67; H04Q 11/00

(52) U.S. Cl. CPC H04L 12/12; G16H 40/67; H04Q 11/0067; H04Q 2011/0083; H04Q 2011/0088

(57) ABSTRACT

There is herein described a method of preventing a monitoring network entering a low-power state, the monitoring network comprising a head end and a remote end, the remote end comprising a sensor adapted to sense a parameter, the method comprising: In response to the sensor sensing a change in the parameter, transmitting, from the remote end to the head end, an indication that the sensor has sensed the change in the parameter; At the head end, in response to receiving the indication that the change has been sensed by the sensor, transmitting an instruction to the remote end not to enter a low-power state.

Remote healthcare settings are often provided with a monitoring network. The monitoring network is typically an Internet of Things (IoT) network and has a head end and a remote end, the remote end being located in the premises of the person requiring care. The person requiring care could be, e.g, a patient, an elderly person or a person with special needs. A Passive Optical Network (PON) connects the head end and the remote end. The IoT network is managed by a centrally-located management service platform. The management service platform is adapted to manage multiple such IoT networks. The remote end of the IoT network typically has an ONU which connects to multiple sensors and actuators. Sensors include heart rate sensors and motion sensors.

If the monitoring network detects that the patient requires assistance, the monitoring network can take remedial action. In particular, if, e.g. a motion sensor in the monitoring network sends a signal to the head end indicating the patient has fallen over and is not moving, the service management platform responds by sending an instruction, via the ONU, to an alarm actuator to call an ambulance.

In order to conserve energy, the monitoring network can be put into a low-power state on a time cycle controlled by the PON management. This typically involves turning off the ONU's transmitter and/or receiver for a number of hours, e.g. overnight.

It is desirable to provide an improved monitoring network which is flexible enough to respond quickly to a patient's needs.

According to a first aspect of the invention there is provided a method of preventing a monitoring network entering a low-power state, the monitoring network comprising a head end and a remote end, the remote end connecting to a sensor adapted to sense a parameter, the method comprising:
In response to the sensor sensing a change in the parameter, transmitting, from the remote end to the head end, an indication that the sensor has sensed the change in the parameter;
At the head end, in response to receiving the indication that the change has been sensed by the sensor, transmitting an instruction to the remote end not to enter a low-power state.

When a system is in a low-power state, it can take several seconds to “wake-up” the system so that it is fully operational. This normally involves the ONU switching on its receiver and/or transmitter. In remote-care settings the need to obtain assistance for a patient can be time-critical. The present invention addresses this by ensuring that once physical movement of the patient, or another factor, has been sensed, the system remains in a fully operation state, at least for a period of time. This enables the system to react quickly to further developments.

The monitoring network may be a PON. The remote end may comprise an Internet of Things (IoT) network. The parameter may be one or more of the following: physical motion, heart rate, blood pressure, body temperature, ambient temperature, ambient light level. The IoT network may be directed to the purpose of remote healthcare. The IoT network may be located in the residence of a patient or an elderly person.

The method may further comprise transmitting, from the head end to the remote end, an instruction to actuate one or more actuators. The one or more actuators may be for one or more of the following purposes: operating a light switch, opening a window, actuating an alarm, calling an ambulance. The network may have a plurality of sensors and a plurality of actuators.

The step of transmitting, from the remote end to the head end, an indication that the sensor has sensed the change in the parameter may be performed by an ONU at the remote end. The indication that a change has been sensed by the sensor may be received by an OLT at the head end. The IoT network may be controlled by service management platform which may be located remotely from the network. The service management platform may further control one or more further IoT networks.

The instruction may be a PLOAM message. The network may comprise a software-defined networking controller. The software-defined networking controller may instruct the head end to transmit the instruction to the remote end. In particular, the software-defined networking controller may instruct the OLT to transmit the instruction to the ONU. The software-defined networking controller may be located at the head end. The software-defined networking controller may be under the control of the service management platform. Unlike in known systems, the service management may instruct the PON management to instruct the ONU not to enter the low-power state.

The ONU may connect to the sensor via a home gateway. The home gateway may be adapted to communicate with the sensor by wireless communication. The wireless communication may be Wi-Fi.

The low-power state may be a state in which the PON ONU consumes less energy than when in normal operation. The low-power state may be one in which an optical transmitter at the ONU is switched off. Alternatively, the low-power state may be a state in which both an optical transmitter and an optical receiver at the ONU is switched off. In normal low power mode operation, the network may enter and exit the low-power state on a time-cycle. During active operation, the ONU optical transceiver and receiver may be operational all the time.

According to a further aspect of the invention there is provided a monitoring network, the monitoring network being adapted to prevent itself entering a low-power state, the monitoring network comprising a head end and a remote end, the remote end connecting to a sensor adapted to sense a parameter, the method comprising:
The remote end being adapted to, in response to the sensor sensing a change in the parameter, transmit to the head end an indication that the sensor has sensed the change in the parameter;
The head end being adapted to, in response to receiving the indication that the change has been sensed by the sensor, transmit an instruction to the remote end not to enter a low-power state.

An embodiment of the invention will now be described in detail, for illustration only, with reference to the appended drawings, in which:

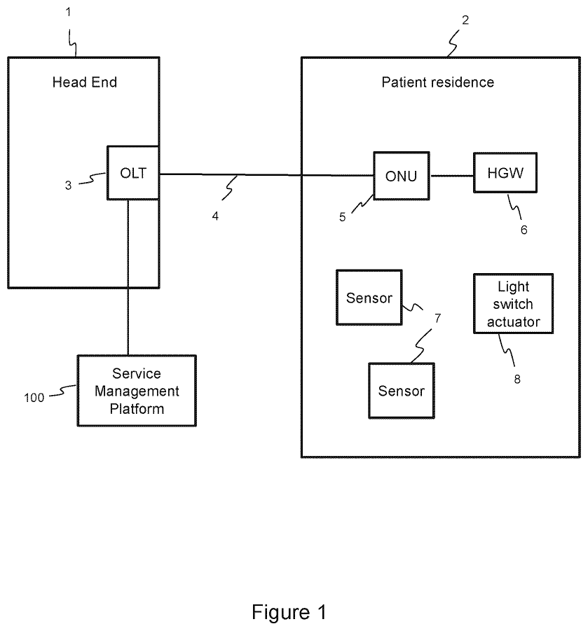

FIG. 1 is a schematic view of an IoT network performing a variety of functions, connected through a PON network to the Service Management Platform, according to the prior art;

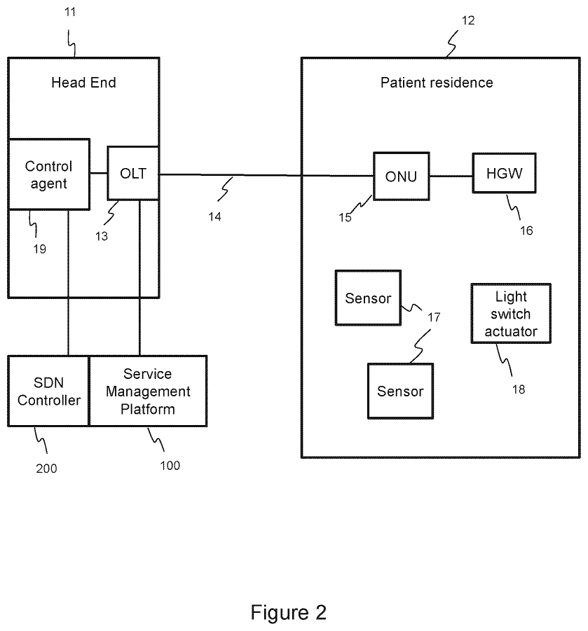

FIG. 2 is a schematic view of an IoT network performing a variety of functions, connected through a PON network to the Service Management Platform, according to the invention;

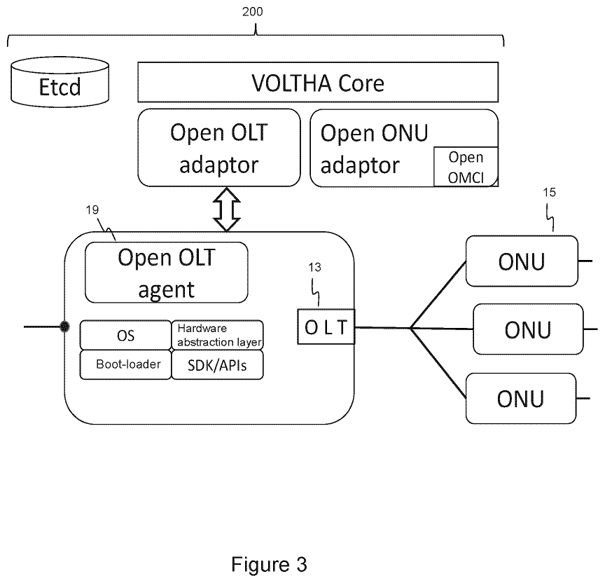

FIG. 3 is an exploded view of the head end of the PON network with SDN capability of FIG. 2;

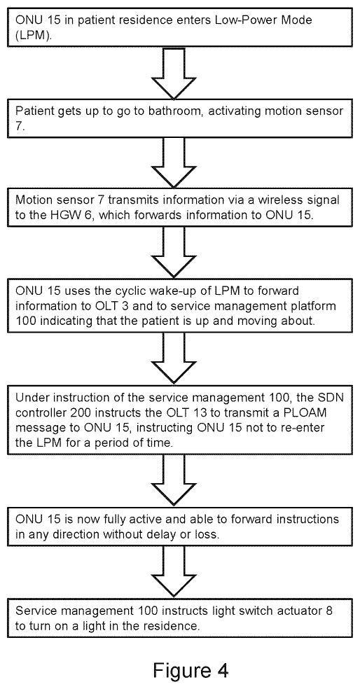

FIG. 4 is a flow chart showing the main steps of the method according to the invention.

FIG. 1 shows an example of an arrangement according to the prior art. The arrangement is a remotely managed IoT system for home healthcare. In particular, an OLT 3 is provided at the head end 1 of a PON. The head end 1 connects to an ONU 5 located at the residence 2 of a patient or elderly person by optical fibre 4. The ONU 5 is connected by ethernet cable to home gateway 6. The patient residence 2 is also provided with sensors 7 and a light switch actuator 8. One of the sensors 7 is a motion sensor for detecting motion of the patient. The other sensor 7 is a heart-rate monitor. A centrally-located service management platform 100 is connected to the OLT 3.

An example of the system in use will now be described. A motion sensor 7 in the patient residence 2 senses motion of the patient. The motion sensor transmits a wireless signal indicating this sensed motion to the home gateway 6. The home gateway 6 transmits an optical signal to the ONU 5, which in turn transmits an optical signal over the PON to the OLT 3 at the head end 1. The OLT 3 transmits a signal to the service management platform 100. In response, the service management platform 100 sends an instruction signal, via the OLT 3, the PON and the ONU 5 to the home gateway 6. The home gateway 6 transmits the instruction signal wirelessly to light switch actuator 8. The signal instructs to the light actuator 8 to turn on the bedroom light. As can be seen from this description, the service management 100 responds to outputs of the sensors 7 by instructing one or more actuators 8 to perform their function. Although not shown, further actuators are provided, such as window openers, alarm actuators etc.

In the interests of reducing energy consumption, the ONU 5 is placed into a low power mode (LPM) at one or more periods during a 24 hour cycle. These periods generally include the time that the patient is asleep. The ONU enters and exits the LPM in accordance with a time cycle. Entering the LPM means the ONU 5 either turns off its transmitter or turns off both its transmitter and its receiver. When the ONU receives local traffic information that it needs to transmit to the OLT during the periods in which the ONU is exercising the LPM, it automatically switches on its optical receiver and transmitter, and waits for the opportunity to transmit the information to the OLT. If, e.g. the motion sensor 7 senses that the patient has gotten up in the night, the motion sensor sends a signal to the ONU 5. This “wakes up” the ONU 5, a procedure which takes one or more seconds. The ONU 5 then transmits a signal to the OLT 3 in the manner described above, after which the ONU will turn off again its optical transmitter and maybe its optical receiver as it is still exercising the LPM.

FIG. 2 shows a schematic drawing of an arrangement in accordance with an embodiment of the invention. The features are largely identical with the features of FIG. 1. However, in FIG. 2 has a Software-Defined Networking (SDN) controller 200. The SDN controller is located centrally, in the vicinity of the service management platform 100. The SDN controller 200 connects to the OLT 3 via a control agent 19 located at the head end 1. The SDN controller is capable of providing an input signal to the OLT 13 via the control agent 19.

A more detailed view of the head end of FIG. 2 is shown at FIG. 3. In FIG. 3 the components of the SDN controller 200 are set out. These components are known in the art and so will not be described in detail here.

An example of the system of FIG. 2 in use will now be described. Consider the case in which the IoT network system of the invention is located in the residence of an elderly person. During the night the ONU 15 is in LPM. If the elderly person gets up to go to the bathroom, motion sensor 17 will be activated. Motion sensor 17 transmits a signal, via home gateway 16 to ONU 15. This signal causes ONU 15 to wake up after a delay. Once woken, ONU 15 transmits a signal to OLT 13 over the PON. The signal is relayed to the SDN controller 200 via control agent 19 and then on to the service management 100. In response, service management 100 instructs the SDN controller 200 to instruct the OLT 13 to send a PLOAM (Physical Layer Operation, Administration and Maintenance) message to ONU 15. The PLOAM message may be, e.g. “Sleep_Allow (OFF)”. This instructs ONU 15 not to re-enter LPM for a period of time, unless otherwise instructed. After that period has elapsed, the ONU 15 re-enters LPM. During the period, if the sensors 17 sense further activity or other issues that require action, the ONU 15 will be able to take such action without needing time to wake up its optical transmitter and receiver. This improves the reaction time of the system in what may be a time-critical situation.

Once it has instructed the ONU 15 not to re-enter LPM, the service management 100 instructs the IoT system to take action to assist the resident. In particular, to instruct light switch actuator 18 to turn on a light in the residence. The ONU 15 is active sending information both ways without traffic packets delay or loss.

A flow chart showing the main steps of the method is provided at FIG. 4.

1. A method of preventing a monitoring network entering a low-power state, the monitoring network comprising a head end and a remote end, the remote end connecting to a sensor adapted to sense a parameter, the method comprising:
In response to the sensor sensing a change in the parameter, transmitting, from the remote end to the head end, an indication that the sensor has sensed the change in the parameter;
At the head end, in response to receiving the indication that the change has been sensed by the sensor, transmitting an instruction to the remote end not to enter a low-power state.

2. A method as claimed in claim 1, wherein the parameter is one of: physical motion, heart rate, blood pressure, body temperature, ambient temperature, ambient light level or a security breach.

3. A method as claimed in claim 1, wherein the monitoring network is for the purpose of remote healthcare.

4. A method as claimed in claim 1, wherein the remote end of the monitoring network is located in the residence of a patient or an elderly person.

5. A method as claimed in claim 1, wherein the method further comprises, in response to receiving the indication that the change has been sensed by the sensor, transmitting, from the head end to the remote end, an instruction to actuate one or more actuators.

6. A method as claimed in claim 5, wherein the one or more actuators are for one or more of the following purposes: operating a light switch, opening a window, actuating an alarm, calling an ambulance.

7. A method as claimed in claim 1, wherein the sensor is part of an IoT network.

8. A method as claimed in claim 1, wherein the monitoring network comprises a Passive Optical Network.

9. A method as claimed in claim 1, wherein the monitoring network comprises a software-defined networking controller.

10. A method as claimed in claim 1, wherein the instruction to the remote end not to enter a low-power state is a PLOAM message.

11. A method as claimed in claim 1, wherein the low-power state is a state in which the IoT network consumes less energy than when in normal operation.

12. A method as claimed in claim 1, wherein the low-power state is a state in which a transmitter at the ONU is switched off.

13. A method as claimed in claim 1, wherein the low-power state is a state in which a transmitter at the ONU is switched off.

14. A method as claimed in claim 1, wherein the network enters and exits the low-power state on a time-cycle.

15. A monitoring network, the monitoring network being adapted to prevent itself entering a low-power state, the monitoring network comprising a head end and a remote end, the remote end connecting to a sensor adapted to sense a parameter, the method comprising:
The remote end being adapted to, in response to the sensor sensing a change in the parameter, transmit to the head end an indication that the sensor has sensed the change in the parameter;
The head end being adapted to, in response to receiving the indication that the change has been sensed by the sensor, transmit an instruction to the remote end not to enter a low-power state.
